(19) United States
(12) Patent Application Publication
(10) Pub. No.: US 2025/0367378 A1
(43) Pub. Date: Dec. 4, 2025

(54) AUTOMATED MEDICAMENT DELIVERY DEVICES, CONTROLLERS, AND METHODS FOR INCORPORATING EXTENDED BOLUS SIGNALS WITHIN AUTOMATED MEDICAMENT DELIVERY ALGORITHMS

(71) Applicant: Insulet Corporation, Acton, MA (US)

(72) Inventors: Joon Bok Lee, Acton, MA (US); Yibin Zheng, Hartland, WI (US); Ashutosh Zade, San Diego, CA (US); Jason O'Connor, Acton, MA (US)

(21) Appl. No.: 19/220,954

(22) Filed: May 28, 2025

Related U.S. Application Data
(60) Provisional application No. 63/654,782, filed on May 31, 2024.

(51) Int. Cl. A61M 5/172

(52) U.S. Cl. CPC A61M 5/172; A61M 5/1723; A61M 2205/3379; A61M 2230/201

(57) ABSTRACT

A method for incorporating an extended bolus signal is disclosed. The method includes determining a target dose amount of medicament to deliver for a control cycle. The method also includes obtaining data related to an extended bolus. The method further includes determining an additional medicament amount to deliver during the control cycle corresponding to the extended bolus. The method yet further includes determining a total medicament delivery amount for the control cycle corresponding to the target dose amount and the additional medicament amount. The method still further includes sending a command to a delivery mechanism to cause delivery of the total medicament delivery amount for the control cycle.

### CROSS-REFERENCE TO RELATED APPLICATIONS

This application claims the benefit under 35 U.S.C. § 119 (e) of U.S. Provisional Patent Application Ser. No. 63/654,782, filed May 31, 2024, the disclosure of which is hereby incorporated herein in its entirety by this reference.

### TECHNICAL FIELD

The present disclosure generally relates to medicament delivery systems. More particularly, the present disclosure relates to automated medicament delivery devices, controllers, and methods for incorporating extended bolus signals within automated medicament delivery algorithms.

### BACKGROUND

Automated medicament delivery devices (AMD, e.g., Automated Insulin Delivery (AID) device, without limitation) are often used to administer medicaments to the body of a patient via a cannula inserted into the body to treat medical conditions (e.g., Type 1 Diabetes, without limitation).

A bolus of medicament (e.g., a correction bolus or a carbohydrate bolus) may be delivered by the AMD to the user-body as an immediate bolus (a specified amount of medicament administered in a single dose), an extended bolus (a specified amount of medicament administered as a sequence of discrete doses at a constant rate over a set duration of time), or a combination bolus (a portion of a specified amount of medicament administered immediately in a single dose and the remainder administered over a set duration of time).

In the case of insulin therapy, users may extend administration of a bolus of medicament over longer periods to account for meals with slow absorption times, such as 50% of the medicament delivery being executed over the next 30 minutes at a constant rate. Such extended boluses are typically disabled while AMDs are being utilized, given that the AMD algorithm is able to deliver medicament above the user's basal amount and compensate for changes in analyte readings for the user-body or slow glucose excursions (after a meal for a user with type 1 Diabetes) accordingly. When use of an extended bolus is desired, deactivation of the automated delivery of the basal amount of medicament may occur.

### BRIEF SUMMARY

In one illustrative embodiment, the present disclosure provides a method. The method includes determining a target dose amount of medicament to deliver for a control cycle. The method also includes obtaining data related to an extended bolus. The method further includes determining an additional medicament amount to deliver during the control cycle corresponding to the extended bolus. The method yet further includes determining a total medicament delivery amount for the control cycle corresponding to the target dose amount and the additional medicament amount. The method still further includes sending a command to a delivery mechanism to cause delivery of the total medicament delivery amount for the control cycle.

In another illustrative embodiment, the present disclosure provides an automated medicament delivery device. The automated medicament delivery device includes one or more processors and memory. The memory includes instructions that, when executed, cause the one or more processors to: determine a target dose amount of medicament to deliver for a control cycle; obtain data related to an extended bolus; determine an additional medicament amount to deliver during the control cycle corresponding to the extended bolus; determine a total medicament delivery amount for the control cycle corresponding to the target dose amount and the additional medicament amount; and deliver the total medicament amount during the control cycle utilizing an automated medicament delivery system.

In a further illustrative embodiment, the present disclosure provides a process. The process includes executing an algorithm, for controlling medicament delivery, without an extended bolus signal to determine a target dose amount. The process also includes comparing the target dose amount to a threshold value. The process further improves in response to the target dose amount being greater than the threshold value, adding an extended bolus amount to the target dose amount and sending a final recommendation to a delivery system. The process yet further includes in response to the target dose amount being less than the threshold value, adding the extended bolus signal to an input basal amount for an input to the algorithm, executing the algorithm with the extended bolus signal included in the input, and sending an algorithm recommendation to the delivery system.

In another illustrative embodiment, the present disclosure provides a controller. The controller includes one or more processors and memory. The memory includes instructions that, when executed, cause the one or more processors to: execute an algorithm, for controlling medicament delivery, without an extended bolus signal to determine a target dose amount; compare the target dose amount to a threshold value; in response to the target dose amount being greater than the threshold value, add an extended bolus amount to the target dose amount and sending a final recommendation to a delivery system; and in response to the target dose amount being less than the threshold value, add the extended bolus signal to an input basal amount for an input to the algorithm, executing the algorithm with the extended bolus signal included in the input, and sending an algorithm recommendation to the delivery system.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a schematic diagram illustrating a medicament delivery system in accordance with embodiments;

FIG. 2 is a block diagram of a medicament delivery system 200 for controlled administering of medicament, in accordance with one or more examples;

FIG. 3 is a flowchart of a method for delivering an extended bolus of medicament in accordance with one or more embodiments;

FIG. 4 is a flowchart of a method for applying an offset to safety constraints; and

FIG. 5 is a flowchart of a process for delivering an extended bolus of medicament in accordance with one or more embodiments.

### DETAILED DESCRIPTION

In various embodiments, medicament delivery systems, and in particular, automated medicament delivery devices and controllers, and methods for incorporating an extended bolus into delivery of medicament are disclosed.

As will be described in detail below, the methods may include determining a total medicament amount to be delivered by including a target dose amount in units for each control cycle combined with an additional medicament delivery in units corresponding to the extended bolus. The additional medicament delivery may only be delivered if the target dose of medicament is above a threshold value The additional medicament delivery corresponding to the extended bolus for each control cycle may be limited by a determined maximum amount of medicament delivery possible for a current control cycle, which may be bounded by the current basal medicament delivery rate.

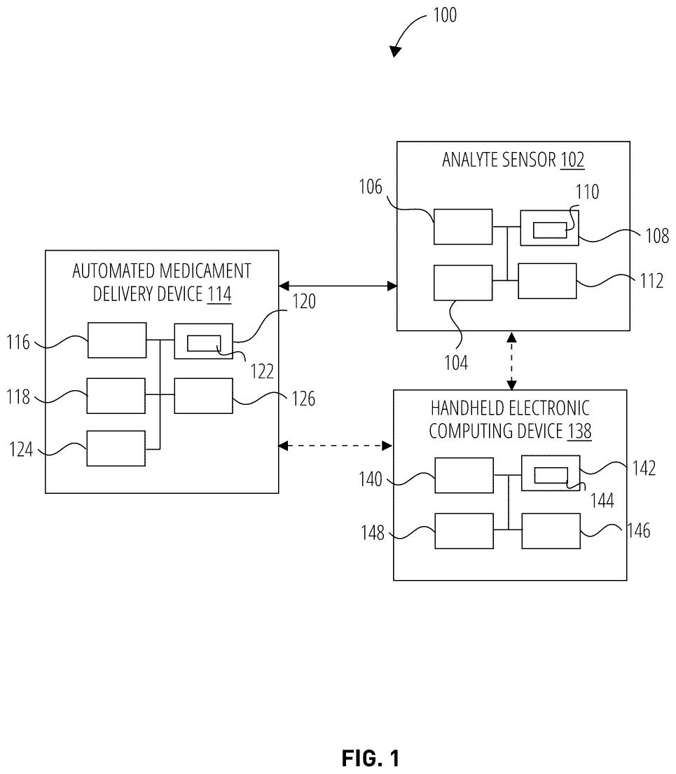

FIG. 1 is a schematic diagram illustrating a system 100 for automated administration of medicament to a user-body, in accordance with one or more embodiments.

In one or more embodiments, system 100 may be capable of one or more operative modes of administration of medicament. Non-limiting examples of the one or more operative modes include: fully automated administration of medicament, partially automated administration of medicament, or manual administration of medicament. In one or more embodiments, system 100 may be capable of alternating between multiple (e.g., two or more, without limitation) operative modes. As a non-limiting example, system 100 may alternate between one or more of: fully automated operation, partially automated operation, and manual operation.

System 100 may administer medicament at least partially based on one or more values representative of amounts of one or more analytes present within a user-body (such values are, respectively, an “analyte value”). The one or more analytes may include constituents of the user-body and foreign substances, such as medicaments, markers, metabolites, and combinations or subcombinations of one or more of the foregoing, without limitation. The system 100 may also administer an amount of medicament at least partially based on user inputs (e.g., a user-defined bolus amount or details related to a meal consumed or about to be consumed, such as number of carbohydrates, amount of fat, and amount of protein, without limitation).

Non-limiting examples of medicaments administrable by system 100 include: insulin, glucagon-like peptide-1 receptor agonist (GLP-1), glucose-dependent insulinotropic polypeptide (GIP), pramlintide, or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated.

System 100 includes an analyte sensor 102 and an automated medicament delivery device 114. System 100 may optionally include a handheld electronic computing device 138.

The analyte sensor 102 is configured to obtain data related to one or more analytes within the user-body (“analyte data”). In various embodiments, the analyte data may include one or more analyte values. In various embodiments, the analyte sensor 102 is an analytical bio-sensing device, such as a continuous glucose monitor (CGM) or an integrated continuous glucose monitor (ICGM) (e.g., examples of commercially available analytical bio-sensing devices include the FREESTYLE LIBRE® 3 manufactured by Abbott or the DEXCOM® G6 manufactured by Dexcom, without limitation).

The analyte sensor 102 includes a filament 104 and various electronic components. The filament 104 is configured to obtain data related to one or more analytes within a user-body and provide the data to the various electronic components of the analyte sensor 102. The filament 104 may be configured to obtain the data directly from fluids of a user-body, including, without limitation, interstitial fluids of a user-body, from tissue of a user-body, combinations thereof, or in any other manner known in the art.

The various electronic components of the analyte sensor 102 include one or more processors 106, a memory 108, and communication equipment 112. Instructions 110 include instructions for processing data obtained via the filament 104. When the instructions 110 are executed by the one or more processors 106, the instructions 110 cause the one or more processors 106 to process the data obtained via the filament 104. The instructions 122 may be implemented in hardware (e.g., one or more hardware processors of the one or more processors 106, such as an integrated circuit, application specific integrated circuit (ASIC), digital signal processor (DSP), or other logic circuit, without limitation), implemented in software (e.g., firmware, software, machine code, applications, without limitation), or a combination thereof. The instructions for processing the data obtained via the filament 104 may include one or more instructions respectively for determining analyte values at least partially based on the data, or for sending the data, analyte values or both to the automated medicament delivery device 114 and/or the handheld electronic computing device 138.

The communication equipment 112 is configured to facilitate communication (e.g., a device or interface for wired communication, wireless communication, both wired and wireless communication, without limitation) of the analyte sensor 102 with other devices, including the automated medicament delivery device 114 and/or the handheld electronic computing device 138, without limitation. Such communication may be according to any appropriate wired or wireless communication protocol, such as WI-FI®, BLUETOOTH®, near-field communication (NFC), radio-frequency identification (RFID), or any other radio-frequency, infrared, or optical communication technology.

The automated medicament delivery device 114 is configured to administer medicament to a user-body, such as subcutaneously into the user-body, without limitation, in accordance with one or more embodiments. In one or more embodiments, the automated medicament delivery device 114 may offer one or more operative modes for administration of medicament to a user-body. When operating in some of the operative modes, automated medicament delivery device 114 may administer medicament at least partially responsive to analyte values, including, without limitation, analyte values received from analyte sensor 102. When operating in some further operative modes, automated medicament delivery device 114 may administer medicament at least partially responsive to user input. When operating some yet further operative modes, automated medicament delivery device 114 may administer medicament at least partially responsive to analyte values and user input. Non-limiting examples of the one or more operative modes offered by automated medicament delivery device 114 include: fully automated administration of medicament, partially automated administration of medicament, or manual administration of medicament. When operating in an operative mode that includes manual administration of medicament, automated medicament delivery device 114 may administer medicament solely in response to a user input (e.g., delivers medicament in response to a user confirmation of delivery of medicament or in response to a user instruction to delivery medicament, without limitation). When operating in an operative mode that includes fully automated administration of medicament, automated medicament delivery device 114 may administer medicament solely in response to analyte values (e.g., delivers medicament in response to one or more analyte values, without limitation). When operating in an operative mode that includes partially automated administration of medicament, automated medicament delivery device 114 may administer medicament in response to analyte values and user input (e.g., delivers medicament in response to a user input and an analyte value, or alternately delivers medicament in response to a user input or in response to analyte values, without limitation). Medicament administration may include administration of a basal amount of medicament regularly delivered a control interval (e.g., at a determined basal rate, without limitation) to keep analyte levels stale and within a determined or predetermined range. Medicament administration may also include administration of bolus amounts of medicament administered as an immediate bolus, an extended bolus, or a combination bolus (combination of an immediate bolus and an extended bolus). The bolus amount of medicament may be a correction bolus responsive to a change in analyte levels or a user-defined bolus (e.g., responsive to user inputs provided, such as a user-defined bolus amount or details related to a meal consumed or about to be consumed, such as number of carbohydrates, amount of fat, and amount of protein, without limitation).

The automated medicament delivery device 114 includes a delivery system 116, one or more processors 118, memory 120, communication equipment 124, and a power source 126. In one or more embodiments, the automated medicament delivery device 114, or portions thereof, may be a wearable device and may be secured to a user-body (e.g., secured via one or more adhesive layers attaching the automated medicament delivery device 114 to the skin of the user-body or a material that is secured to the user-body, without limitation).

In various embodiments, the delivery system 116 is configured to cause an amount of medicament to move (e.g., flow, without limitation) toward and/or into a user-body.

In various embodiments, delivery system 116 may deliver amounts of medicament at least partially responsive to requests. In various embodiments, instructions 122 of memory 120 may include instructions for determining and generating requests for delivery system 116. In various embodiments, instructions 122 may include instructions for determining one or more amounts of medicament, determining a timing for delivery of one or more amounts of medicament, and for generating one or more requests for delivery system 116 related to the same. When such instructions of instructions 122 are executed by one or more processors 118, the one or more processors 118 determine the amounts of medicament and timing of delivery, generate requests for the delivery system 116 at least partially based on the determined amounts and timing, and provide the requests to delivery system 116.

The communication equipment 124 is configured to facilitate communication (e.g., wireless communication, without limitation) of the automated medicament delivery device 114 with other devices, including, without limitation, communication between analyte sensor 102 and the automated medicament delivery device 114. The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In various embodiments, the communication equipment 124 includes an Internet of Things (IoT) Subscriber Identity Module (SIM) card (e.g., a machine-to-machine SIM card, a Universal Integrated Circuit Card, without limitation).

The power source 126 is configured to supply power to the delivery system 116 and the various electronic components, such as the one or more processors 118, memory 120, communication equipment 124, and the like. Power source 126 may be, as a non-limiting example, a power storage device (e.g., a battery, without limitation), a power inlet, a power regulator, or combination thereof.

In various embodiments, the handheld electronic computing device 138 is configured to communicate with the automated medicament delivery device 114 and the analyte sensor 102. The handheld electronic computing device 138 may be chosen from among a dedicated electronic device, a smart phone, a tablet computer, a wearable device (e.g., a smart watch, without limitation), a cloud computing device, and the like.

The handheld electronic computing device 138 may include one or more processors 140, memory 142 that stores instructions 144 to be executed by the one or more processors 140, communication equipment 146, and a user interface 148. The one or more processors 140 and memory 142 may be configured/programmed to perform any of the operations discussed above, as well as other control operations for managing the automated medicament delivery device 114 and the analyte sensor 102.

The communication equipment 146 is configured to facilitate communication (e.g., wireless communication, without limitation) of the handheld electronic computing devices 138 with other devices, such as the automated medicament delivery device 114 and the analyte sensor 102. The communication may be wired or wireless communication, such as via a wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In some of these embodiments, the automated medicament delivery device 114 and the handheld electronic computing devices 138 are paired via the short-range wireless protocol (e.g., paired via BLUETOOTH®, without limitation), and successful message transmissions between the automated medicament delivery device 114 and the handheld electronic computing devices 138 may be acknowledged.

The user interface 148 is configured to provide a user with information and obtain information from the user via one or more of a display, an audio speaker, an LED, a vibration motor, a button (e.g., a mechanical button, capacitive button, without limitation), a gesture-based interface, and the like.

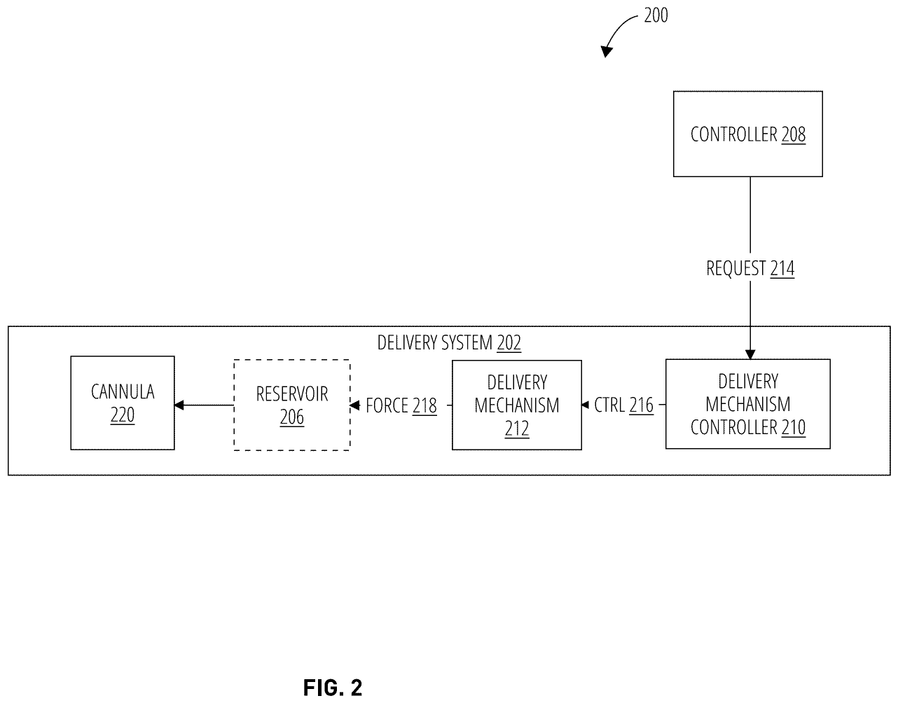

FIG. 2 is a block diagram of a medicament delivery system 200 for controlled administration of medicament to a user-body, in accordance with one or more examples.

The controller 208 is configured to manage automated medicament delivery device 114 and, more generally, administration of medicament to a user-body. In one or more embodiments, controller 208 may be implemented by instructions 122 and one or more processors 118 of automated medicament delivery device 114 of FIG. 1.

In various embodiments, controller 208 and delivery system 202 may be realized in different devices (e.g., controller 208 may be realized in a physically different device (or devices) than delivery system 202 is realized, such as the handheld electronic computing device 138, without limitation), or in the same device. When realized in different devices, functionality of controller 208 and delivery system 202 may be implemented, at least in part, by respective memory and one or more processors of their respective devices. When realized in a same device, functionality of controller 208 and delivery system 202 may be implemented, at least in part, by memory and one or more processors, respective memory and respective one or more processors, or a combination thereof. Non-limiting examples of devices in which controller 208, or a portion thereof, may be realized include: a handheld electronic computing device, such as a dedicated electronic device, a smart phone, a tablet computer, a wearable device (e.g., a smart watch, without limitation), a cloud computing device, and the like.

In various embodiments, the controller 208 may be configured to receive analyte data (e.g., from the analyte sensor 102, without limitation) including analyte values. In one or more embodiments, controller 208 may determine information about analytes within a user-body at least partially based on analyte data, for example, amounts, trends, distributions, without limitation. The controller 208 may analyze information about analytes in a user-body and may present the information and/or analysis to a patient, caregiver, or healthcare provider, as a non-limiting example, via an application (e.g., executed on a personal computer, smart phone, cloud server, or combinations thereof).

In various embodiments, the controller 208 may be configured to receive information from inputs from the patient or a caregiver (e.g., when the patient ate a meal or when the patient exercised, without limitation), and inputs from other electronic devices (e.g., information from a smartwatch, without limitation) and to utilize such information as discussed herein. For example, in various embodiments, controller 208 may utilize some or a totality of such information to determine amounts of medicament to administer and timing of administration of medicament. Further, controller 208 may also be configured to determine requests, including a request to administer dose 214, and send those requests to the automated medicament delivery device 114.

In various embodiments, controller 208 may be configured to determine a target dose amount to administer to a user of medicament delivery system 200. Controller 208 may determine a target dose amount at least partially based on therapy parameters, meal information, analyte values, and a control algorithm, without limitation.

In the context of insulin therapy to treat diabetes, therapy parameters may include insulin sensitivity factor (ISF), carbohydrate ratio (CR), amount of daily dose of long-acting insulin (LAI), doses of fast-acting or rapid-acting insulin, a current glucose value, and derivatives thereof without limitation. The timing and target dose amounts associated with requests generated by controller 208 may be governed by one or more control algorithms, discussed below.

Controller 208 may send a request to administer dose to delivery system 202, and more specifically, delivery mechanism controller 210. The request to administer dose may include the target dose amount determined by controller 208.

The cannula 220 is insertable into a user-body (e.g., with a tip thereof positioned subcutaneously, without limitation) and is configured to provide medicament to a user-body (e.g., subcutaneously into the user-body, without limitation).

The reservoir 206 is configured to store and retain a medicament therein. As a non-limiting example, the reservoir 206 may be a hollow body, a flexible pouch, a chamber, or a vial, without limitation. In various embodiments, reservoir 206 is a fluid reservoir for holding medicament and may be, as a non-limiting example, formed from the walls of a cartridge. In the cartridge example, delivery system 202 may include a chamber (i.e., a space or region defined within delivery system 202) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in reservoir 206 will be greater in a pre-filled state than the volume in an exhausted state. Additionally or alternatively to the cartridge example, delivery system 202 is a multi-part delivery device where one of the two parts includes the reservoir 206 and the other one of the two parts includes the delivery mechanism controller 210. The other one of the two parts may optionally further include controller 208. Either one of the two parts may optionally include delivery mechanism 212 (e.g., a pump mechanism, without limitation). The one of the two parts that includes reservoir 206 is disposable (i.e., a “disposable part”) and configured to be removably secured to the other part of medicament delivery system 200. When reservoir 206 is exhausted, the disposable part may be removed and a replacement part including a reservoir 206 optionally in a pre-filled state.

Delivery mechanism 212 is configured to urge fluid in reservoir 206 toward an interface for dispensing fluid (interface not shown). In various embodiments, delivery mechanism 212 may be positioned adjacent to reservoir 206. The delivery mechanism 212 is configured to cause an amount of the medicament to be administered to the user-body by causing the amount to flow from the reservoir 206 toward and into a user-body via cannula 220, which is in fluidic communication with the reservoir 206. In various embodiments, delivery mechanism 212 may utilize any suitable mechanism to generate positive displacement or negative displacement to transfer amounts of medicament from reservoir 206 toward cannula 220 and a user-body. Non-limiting examples of mechanisms include a ratchet gear pump, peristaltic pump, linear peristaltic pump, piston pump, gear pump, bellows pump, or diaphragm pump.

For example, delivery mechanism 212 may apply a force to an urging mechanism (e.g., a plunger, flexible-walled tube, without limitation) free to move within reservoir 206, and via such a force, move the urging mechanism in a direction that urges fluid in reservoir 206 toward the aforementioned interface. In one or more examples, delivery mechanism 212 may include an electrical motor (e.g., an AC or DC motor) that produces a force to, directly or indirectly, move the urging mechanism to perform a delivery action. A delivery action dispenses at a predetermined rate (i.e., a predictable amount of fluid over a predictable duration of time). The delivery mechanism 212 may be capable of multiple rates of delivery, and in one or more examples, may be preconfigured to use a same rate of delivery all the time, or, in some cases, may be provided discretion to determine a rate of delivery consistent with a target dose amount included with a request.

Such an electric motor may be a current controlled electric motor, voltage controlled electric motor, pulse-width controlled electric motor, or combination or sub combination thereof. Such an electronic motor may be directly or indirectly digitally controlled. The control signal 216 may be determined and generated by delivery mechanism controller 210 to correspond to a delivery action. A control signal 216 may also be referred to herein as a “command 216” or an “instruction 216.”

Delivery mechanism controller 210 may generate control signals 216 corresponding to one or more delivery actions at least partially based on a request to administer dose 214 received from controller 208. Control signal 216 may include first control signals to cause delivery mechanism 212 to generate resultant force 218, and a second, different control signal to cause delivery mechanism 212 to not generate or stop generating force 218. Utilizing control signals 216, delivery mechanism controller 210 may control a length of a duration of time that delivery mechanism 212 produces force 218 and applies it to dispense fluid from reservoir 206, and indirectly, an amount of fluid dispensed from reservoir 206.

When delivery mechanism controller 210 generates control signal 216 in response to a request to administer dose 214 from controller 208, it may generate the control signal 216 at least partially based on a value of a target dose amount included with, or indicated by, request to administer dose 214. One or more delivery actions may be utilized to dispense an amount of fluid corresponding to a dose amount determined by controller 208. For example, a fluid amount dispensed according to a delivery action may be less than a dose amount. Generally speaking, the delivery mechanism 212 and delivery system 202 are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more examples, delivery mechanism controller 210 may be configured to determine and generate feedback information about delivery actions, such as times of delivery actions and dispensed amounts, without limitation. Feedback information may be generated based on information generated by delivery mechanism 212 or by sensors utilized by delivery mechanism controller 210 to monitor operation of delivery mechanism 212 (sensors not depicted). For example, sensors to monitor mechanical movement, current consumption, a voltage profile of an electric motor, without limitation. Such information may be logged and provided to and stored at controller 208 or a handheld electronic computing device 138, without limitation; e.g., later processing or reading, without limitation. For example, the logs can be processed to determine patterns that may be utilized to determine whether delivery system 202 is operating as expected (e.g., in a predictable manner, without limitation), and if a difference between actual and expected operation exceeds a threshold, delivery mechanism controller 210 may be updated (e.g., firmware, parameters, or both, of delivery mechanism controller 210 may be updated, without limitation) to compensate or correct for the difference. Additionally or alternatively to updating the firmware or parameters, in a multi-part system, one or more parts including delivery mechanism controller 210 may be indicated as needing replacement (e.g., an alarm or alert is generated at delivery system 202, medicament delivery system 200, a mobile device or computer in communication therewith, without limitation).

### Control Algorithm Architecture

Values of target dose amounts and timing of requests to administer the same generated by controller 208 may be governed by one or more control algorithms implemented at controller 208. Generally speaking, such a control algorithm may, via one or more control actions, try to cause an amount of analyte in the body (represented by values captured by, or at least partially based on, an analyte sensor or monitor, without limitation) to track a target amount of analyte (in control terms, the target amount of analyte is the “set point”) in the body. The control actions may include amount and timing of administration of doses of medicament that functions as a therapeutic agent in the body.

In one or more examples, a control algorithm may employ a modular design in which core functionality may be separated from dependent functionality. Dependent functionality includes, as non-limiting examples, functionality that may be implementation-specific to a current environment, such as software abstraction for an analyte sensor. Such dependent functionality may include software services which interface with implementation-specific features that affect inputs or outputs to the control algorithm. Dependent functionality may include, as a non-limiting example, functionality for managing algorithm initialization and upload of administration history, managing the control algorithm's state and data variables, and maintaining cycle-to-cycle data utilized by the algorithm such as analyte values, current or historical. Dependent functionality may include functionality responsible for sending requests to administer doses to delivery system 202 that are determined by the control algorithm.

Transmission of data, including, without limitation, request to administer dose 214, may occur over wired, wireless, or a combination thereof, communication paths, in a synchronous or asynchronous manner. In one or more examples, the control algorithm may include one or more layers to provide safety or other operational constraints (e.g., for edge case handling, without limitation).

In one or more examples, a control algorithm may determine a target dose amount that is included with a request, at least partially based on a dynamic model of the body's response (in terms of amount of analyte in the body), for administration of analyte to the body. The control algorithm may determine future amount of analyte or a change in amount of analyte over a predetermined duration of time for a respective dose amount of medicament and compare the determined future amount or change to a target amount or change. The algorithm may determine target dose amounts of medicament according to control intervals that occur according to a predetermined schedule, on-demand, or both. In one or more examples, control intervals may correspond to intervals such as day-night, weeks, days, twenty-four (24) hours, single hours, and sub-intervals of the same, such as 5-minute intervals.

In some cases, the control algorithm may be or include a control algorithm that handles constraints, such as a model-predictive-control (MPC) algorithm. Non-limiting examples of constraints include: upper and lower bounds on analyte levels that can be set to prevent dangerous hypo- or hyperglycemia; medicament delivery rates of delivery system 202 can be constrained to prevent over- or under-dosing; and considerations related to medicament-on-board to, e.g., prevent stacking of medicament doses waiting to work on analyte in the body (e.g., stacking of insulin waiting to work on glucose, without limitation).

One or more examples discussed herein may refer to administering medicament or a medicament therapy to a user or the user-body. Such discussion is intended to encompass examples where medicament or a medicament therapy is administered to a user by automated medicament delivery devices discussed herein, examples where requests to administer doses in accordance with administering medicament or medicament therapy to the user or user-body are generated by a controller and sent to a delivery device, and examples where instructions (e.g., control signals, without limitation) in accordance with dose amounts and timing included with such requests to administer doses are generated by a delivery mechanism controller and sent to a delivery mechanism.

As will be outlined in detail below, in various embodiments, the algorithm administers an extended bolus (e.g., a carbohydrate bolus or a correction bolus, without limitation) with the target dose amount (e.g., a basal amount of medicament, without limitation) regularly delivered at the control interval (also referred to as a control cycle). The extended bolus is a specified amount of medicament administered as a sequence of discrete doses over a set duration of time at a constant rate.

In various examples, an extended bolus may be specified as an increased rate of medicament delivery (e.g., an increase from, or an additional amount on top of, a basal rate of delivery and any correction boluses or extended boluses being administered, without limitation) over a set duration of time. For example, Equation 1 expresses an increase in medicament delivery as the following:

$${M}_{ex} ( i + j ) = \frac{P \cdot {B}_{s}}{T \cdot n} and j = 1 … T \cdot n \qquad equation 1$$

where M_{ez} is the additional medicament delivery in units (U) corresponding to the extended bolus during each control cycle following the current control cycle, i is the identifier for the current control cycle (e.g., number of the current control cycle, without limitation), j is a number of control cycles over which the extended bolus is to occur, Bₛ is a bolus size (e.g., a specified amount of medicament, without limitation), P is a proportion of the bolus that the user wants delivered in an extended fashion expressed as a decimal ratio or percentage (e.g., 1 or 100% when only an extended bolus is administered or 0.25 or 25%, 0.5 or 50%, or 0.75 or 75% for a combination bolus when a corresponding immediate bolus is also administered with the extended bolus, without limitation), T is a time duration of the extended bolus typically in hours but it may be expressed in minutes, seconds or other units of time and n is a number of control cycles per hour (e.g., n=12 for a control cycle of 5 minutes, without limitation).

In various embodiments, the bolus size for a carbohydrate bolus is determined by

$${B}_{s} = \frac{CHO}{{M}_{C}}$$

where CHO is a carbohydrate content of a meal and M_{C} is a mass to carbohydrate ratio (M:C ratio) that specifies the mass of carbohydrate (e.g., how many grams of carbohydrate, without limitation) that is managed by each unit of medicament.

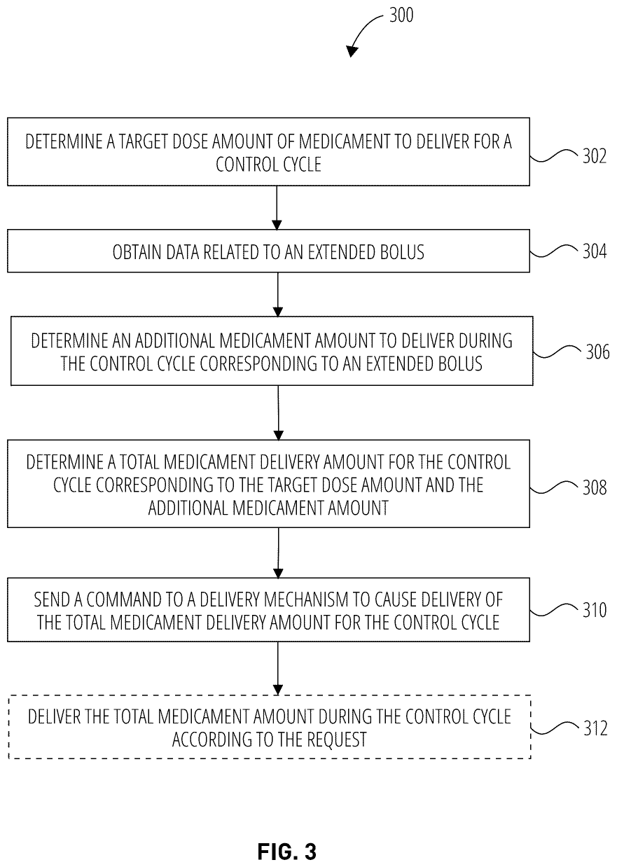

FIG. 3 is a flowchart of a method 300 for delivering an extended bolus of medicament in accordance with one or more embodiments.

The method 300 includes determining a target dose amount of medicament to deliver for a control cycle at act 302. As noted above, the target dose amount may be determined at least partially based on therapy parameters, meal information, analyte values, and a control algorithm, without limitation.

The method 300 also includes obtaining data related to an extended bolus at act 304. The data may include an amount of medicament and a duration of the extended bolus or information related to determining an extended bolus (e.g., a carbohydrate content of a meal, without limitation), which may be used with other relative data for determining the extended bolus (e.g., an M:C ratio that specifies how much weight of carbohydrate are covered by each unit of medicament, without limitation).

The method 300 further includes determining an additional medicament amount to deliver during the control cycle corresponding to the extended bolus at act 306. Determining the additional medicament amount may be performed by any process disclosed herein (e.g., via any of the equations disclosed herein or combinations thereof, without limitation).

The method 300 further includes determining a total medicament delivery amount for the control cycle corresponding to the target dose amount and the additional medicament amount at act 308. In various embodiments, the total medicament delivery amount is determined by adding the target dose amount for the control cycle to the additional medicament amount for the control cycle, where the additional medicament amount is an amount of the extended bolus for the control cycle.

The total medicament amount may be determined as follows:

$${M}_{f} ( i ) = {M}_{ex , f} ( i ) + {M}_{AMD} ( i ) \qquad equation 2$$

where M_{f}(i) is the total medicament delivery in units for each control cycle, M_{ex,f}(i) is the additional medicament delivery in units corresponding to the extended bolus for each control cycle, and M_{AMD}(i) is the target dose amount in units determined by the ADM algorithm.

In various embodiments, the additional medicament delivery corresponding to the extended bolus (M_{ex,f}(i), is only delivered if the AMD algorithm determines to deliver a target dose of medicament above a threshold value. In some of these various embodiments, the additional medicament delivery corresponding to the extended bolus (M_{ex,f}(i) is conditionally delivered as follows:

$${M}_{ex , f} ( i ) = \{ \begin{matrix}{M}_{ex} ( i + j ) & {M}_{AMD} ( i ) \geq TH \\ 0 & {M}_{AMD} ( i ) \leq TH\end{matrix} \qquad equation 3$$

where TH is the threshold value. The threshold value may be the current basal medicament delivery rate for the user, which may be defined as

$$TH = \frac{{M}_{b} ( i )}{n}$$

and M_{b}(i) is the basal amount of medicament delivered per hour. Thus, the threshold value may be the basal amount of medicament delivered per hour divided by the number of control cycles per hour. The average basal amount per hour may be defined as:

$${M}_{b} ( i ) = \frac{TDD}{24} \cdot R$$

where TDD is the Total Daily Dose of medicament and R is a ratio of a basal amount of medicament relative to the TDD (e.g., a percentage of the basal amount of medicament for a basal/bolus balance of the TDD, without limitation). Including a threshold for allowing the extended bolus to be delivered may safeguard a user from receiving too much medicament when the AMD has determined that no medicament or a limited amount of medicament should only be delivered, such as when the AMD algorithm recommends a suspension in medicament delivery.

In various embodiments, the additional medicament delivery corresponding to the extended bolus for each control cycle is limited by a maximum amount of medicament delivery possible that the AMD algorithm recommends for a current control cycle, bounded by the current basal medicament delivery rate. In some of these various embodiments, the extended bolus is defined as:

$${M}_{ex , f} ( i ) = {M}_{ex} ( i + j ) \cdot \frac{{M}_{AMD} ( i )}{{M}_{ub} ( i ) , \frac{{M}_{b} ( i )}{n}} \qquad equation 4$$

where M_{ub} is the maximum medicament delivery that the AMD algorithm may recommend in the current control cycle in units per hour (U/h), bounded by the current basal medicament delivery rate. The extended bolus defined by equation 4 may be utilized for determining the total medicament delivery as defined in equation 2 or for the extended bolus applied while M_{AMD}(i)≥ TH in equation 3.

In various embodiments, the extended bolus is incorporated into an input for the AMD algorithm. In particular, the extended bolus (Mₑₓ(i+j)) is combined with the basal amount of medicament delivered per hour (M_{b}(i)) for the input, which is defined as:

$${M}_{b , input} ( i ) = {M}_{b} ( i ) + {M}_{ex} ( i + j ) \cdot n \qquad equation 5$$

where the extended bolus portion of the input is converted to an hourly amount and added to the standard basal amount of medicament delivered per hour. In these embodiments, acts 302 and 306 may be performed simultaneously. With the extended bolus incorporated into the input for the AMD algorithm with the basal amount of medicament delivered per hour, the AMD algorithm may directly calculate the total medicament delivery (M_{f}(i) for the current control cycle and may dynamically adjust and increase/decrease medicament delivery above the extended bolus through this modification of the input. With the input including the extended bolus, constraints in the AMD algorithm based on the input may scale/increase with the updated input.

In various embodiments, obtaining data related to an extended bolus is provided by a user in a manual input (a total bolus amount, an extended bolus amount, or the carbohydrate content of a meal, without limitation) that is used by the AMD algorithm to incorporate the extended bolus into the medicament delivery utilizing any of the equations outlined above. By incorporating the extended bolus into the AMD algorithm as outlined above, a user may enter the manual input while the system remains operating in an automated mode, while the automated mode accounts for the extended bolus along with accounting for medicament deliveries in previous control cycles (e.g., accounting for medicament on board, without limitation).

In various embodiments, the amount of medicament delivered per control cycle is defined as:

$${M}_{c} ( k ) = \frac{T D D}{24} \cdot R \cdot \frac{1}{n} + \frac{{CHO}_{ex , i} ( k )}{\frac{500}{TDD}} \cdot \frac{1}{{H}_{ex , i} \cdot n} \qquad equation 6$$

where the carbohydrate content of a user's i^{th} extended bolus request CHO (k) is converted into total medicament needed for the extended bolus via the user's M:C ratio and is converted into the amount of medicament delivered per control cycle over a time H_{ex,i} in hours for the extended bolus. In some of these various embodiments, the carbohydrate content and the time for the extended bolus is provided by the user (e.g., via a user interface, without limitation).

Determining the amount of medicament delivered per control cycle in this manner may allow for granular control of the extended bolus by allowing the AMD algorithm to freely determine a proportion of the extended bolus that should be delivered to the user in each discretized dose (e.g., an (additional) amount of medicament to be delivered during each cycle for a period of time).

The method 300 still further includes sending a command to a delivery mechanism to cause delivery of the total medicament delivery amount for the control cycle at act 310. Optionally, the method 300 yet further includes delivering the total additional medicament amount during the control cycle according to the request at act 312. The total medicament amount may be delivered by a delivery mechanism (e.g., the delivery mechanism 212, without limitation).

Allowing the AMD algorithm to determine what fraction of the user's extended bolus should be delivered to the user may impact the AID algorithm's safety profile because the traditional safety constraints in place may be triggered on the one hand, or calculated to be an exaggerated value on the other hand, when a fractional portion of an extended bolus is added to a basal portion of a delivery cycle. The AID algorithm typically utilizes the input basal rate as the basis for determining safety constraints, thresholds, and cutoff amounts. For example, the AID algorithm may utilize a one-time constraint, which is a limit that the AMD algorithm imposes on insulin delivery at a given control cycle may be determined as a multiple of the input basal rate, such as 4 times the basal rate, without limitation. A direct incorporation of the user's extended bolus request into the input basal rate (e.g., summing the basal rate and the extended bolus) may result in the one-time constraint also increasing by 4 times the user's current 5-minute extended bolus request, which could allow undesirable increases to the safety constraint (e.g., an exaggerated value beyond the previous one-time constraint, without limitation) in the insulin delivery possible by the AMD algorithm in the current cycle.

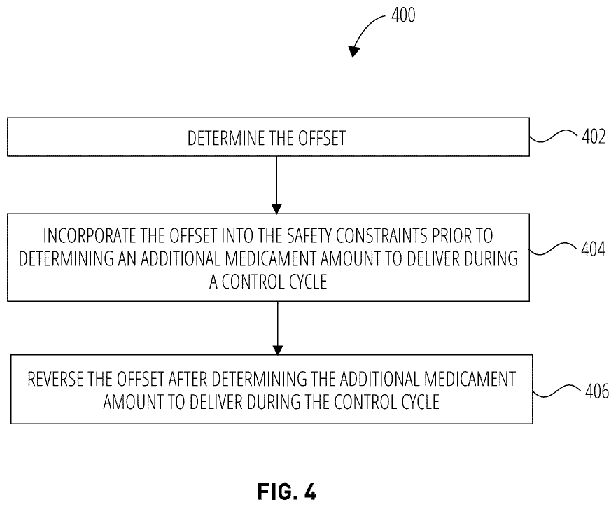

In various examples, such undesirable increases in limits set by safety constraints may be addressed by applying an offset to the safety constraints. FIG. 4 is a flowchart of a method 400 for applying an offset to the safety constraints. The method 400 includes determining the offset at act 402. The offset may be determined for application to all safety constraints and may be equivalent to a determined extended bolus prior to each constraint calculation that incorporates the extended bolus (e.g., prior to act 306 of the method 300, without limitation), and in particular, may be equivalent to the extended bolus for the current control cycle prior to each constraint calculation. The method 400 also includes incorporating the offset into the safety constraints prior to determining an additional medicament amount to deliver during a control cycle at act 404. The offset may be incorporated by adding or subtracting the offset to or from the safety constraints (e.g., safety constraints of the AMD algorithm) applied to the medicament delivery to ensure safe and efficient delivery of the extended bolus with the target dose amount. In various embodiments, the offset is applied to each safety constraint.

The method 400 further includes reversing the offset after determining the additional medicament amount to deliver during the control cycle at act 406 (e.g., after act 306 of the method 300, without limitation). Act 406 may be performed after each respective constraint calculation and may be performed prior to a subsequent control cycle. By subtracting the amount corresponding to the extended bolus proportion, which reverses the offset after each respective constraint calculation, the extended bolus may only impact the raw magnitude of the upper bound constraint.

In some of these various embodiments, the one-time constraint is defined as:

$${U}_{max , OT} = 4 ( {M}_{c} ( k ) - \frac{{CHO}_{ex , i} ( k )}{\frac{500}{TDD}} \cdot \frac{1}{{H}_{ex , i} \cdot n} ) + \frac{{CHO}_{ex , i} ( k )}{\frac{500}{TDD}} \cdot \frac{1}{{H}_{es , i} \cdot n} \qquad equation 7$$

An integral, or total insulin delivery, constraint may also be typically defined as:

$${U}_{max , Int} = {I}_{m} ( {M}_{c} ( k ) - \frac{{CHO}_{ex , i} ( k )}{\frac{500}{TDD}} \cdot \frac{1}{{H}_{ex , i} \cdot n} ) + \frac{{I}_{m}}{T} \frac{{CHO}_{ex , i} ( k )}{\frac{500}{TDD}} \cdot \frac{1}{{H}_{es , i} \cdot n}$$

equation 8
where Iₘ is a total multiple of the integral insulin delivery to be limited and Tis the duration of the integral constraint.

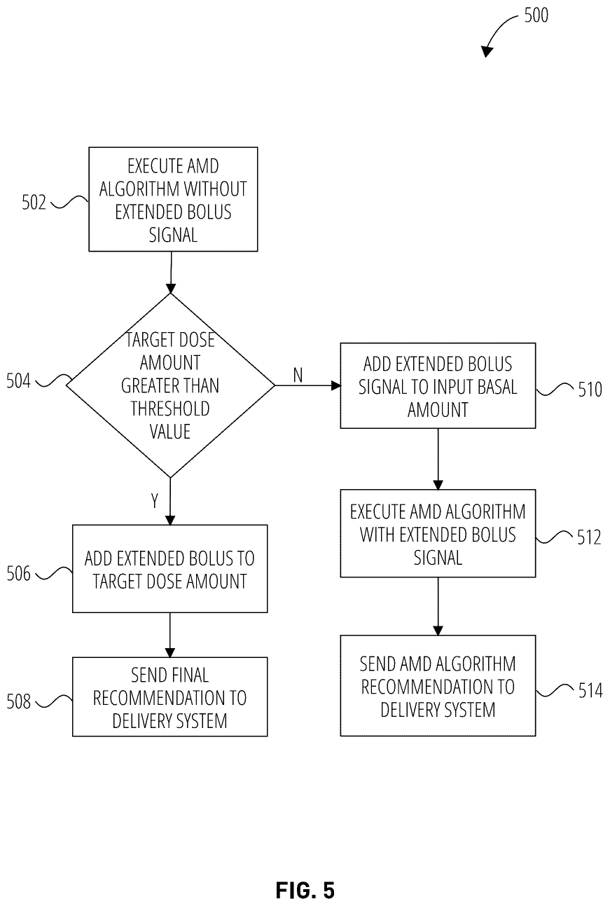

FIG. 5 is a flowchart of a process 500 for delivering an extended bolus of medicament in accordance with one or more embodiments. In various embodiments, the additional medicament amount is added either to the target dose amount after the target dose amount is determined for a particular cycle, or added as an input to the AMD algorithm and the AMD algorithm determines the total medicament amount (the target dose amount combined with the additional medicament amount). The process 500 includes executing the AMD algorithm when in an automated mode without an extended bolus signal to determine a target dose amount (e.g., the AMD algorithm determines the target dose amount without taking into account the extended bolus or prior to receiving the extended bolus signal) at act 502. The extended bolus signal may include the total additional medicament amount and duration for the extended bolus or the data related to the extended bolus for the determination of the additional medicament amount and the duration for delivery thereof.

The process 500 also includes comparing the target dose amount to a threshold value at act 504. Act 504 may be performed in response to receiving the extended bolus signal. The threshold value may be based on the basal amount of medicament. In various embodiments, the threshold value is the value of the basal amount of medicament delivered per cycle

$$( e . g . TH = \frac{{M}_{b} ( i )}{n} ,$$

without limitation). In response to the target dose amount being greater than the threshold value, the process includes adding an extended bolus amount to the target dose amount at act 506 and sending a final recommendation to the delivery system at act 508, the final recommendation including a total medicament delivery target dose amount. In various embodiments, the extended bolus amount is the additional medicament amount of act 306 and the addition is the total medicament delivery amount of act 308 (e.g., the additional medicament amount and total medicament delivery amount determined by any of equations 1˜4 and 6, without limitation).

In response to the target dose amount being less than the threshold value, the process 500 includes adding the extended bolus signal to an input basal amount for an input to the AMD algorithm at act 510 (e.g., equation 5, without limitation), executing the AMD algorithm with the extended bolus signal included in the input at act 512, and sending the AMD algorithm recommendation to the delivery system at act 514. The recommendation causes the delivery system to deliver a recommended amount of medicament to a user.

In various embodiments, the process 500 includes applying an offset to safety constraints (e.g., safety constraints of the AMD algorithm) applied to the medicament delivery to ensure safe and efficient delivery of the extended bolus with the target dose amount. In various embodiments, the offset is applied to all safety constraints. The offset may be equivalent to the extended bolus for the current control cycle prior to each constraint calculation. In some of these various embodiments, the process 500 also includes reversing the offset after each respective constraint calculation.

In the detailed description, reference is made to the accompanying drawings, which form a part hereof, and in which are shown, by way of illustration, specific examples of embodiments in which the present disclosure may be practiced. These embodiments are described in sufficient detail to enable a person of ordinary skill in the art to practice the present disclosure. However, other embodiments may be utilized, and structural, material, and process changes may be made without departing from the scope of the disclosure.

The illustrations presented herein are not meant to be actual views of any particular method, system, device, or structure, but are merely idealized representations that are employed to describe the embodiments of the present disclosure. The drawings presented herein are not necessarily drawn to scale. Similar structures or components in the various drawings may retain the same or similar numbering for the convenience of the reader; however, the similarity in numbering does not mean that the structures or components are necessarily identical in size, composition, configuration, or any other property.

The description may include examples to help enable one of ordinary skill in the art to practice the disclosed embodiments. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the embodiments as generally described herein and illustrated in the drawing could be arranged and designed in a wide variety of different configurations. Thus, the description of various embodiments is not intended to limit the scope of the present disclosure, but is merely representative of various embodiments. While the various aspects of the embodiments may be presented in drawings, the drawings are not necessarily drawn to scale unless specifically indicated.

Furthermore, specific implementations shown and described are only examples and should not be construed as the only way to implement the present disclosure unless specified otherwise herein. Elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

In the Brief Summary and in the Detailed Description, the claims, and in the accompanying drawings, reference is made to particular features (including method acts) of the present disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and embodiments described herein.

Those of ordinary skill in the art would understand that information and signals may be represented using any of a variety of different technologies and techniques. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative methods, logical blocks, modules, and circuits described in connection with the embodiments of the system 100, and in particular, the automated medicament delivery device 114 and the handheld electronic computing device 138, disclosed herein, may be implemented or performed with a general purpose processor, a special purpose processor, a digital signal processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to embodiments of the present disclosure.

The embodiments may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

As used herein, the term “substantially” in reference to a given parameter, property, or condition means and includes to a degree that one skilled in the art would understand that the given parameter, property, or condition is met with a small degree of variance, such as within acceptable manufacturing tolerances. For example, a parameter that is substantially met may be at least about 90% met, at least about 95% met, or even at least about 99% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

As used herein, the terms “adapted,” “configured,” and “configuration” refers to a size, a shape, a material composition, a material distribution, orientation, and arrangement of at least one feature (e.g., one or more of at least one structure, at least one material, at least one region, at least one device) facilitating use of the at least one feature in a pre-determined way.

As used herein, the term “may” with respect to a material, structure, feature, function, or method act indicates that such is contemplated for use in implementation of an embodiment of the disclosure, and such term is used in preference to the more restrictive term “is” so as to avoid any implication that other compatible materials, structures, features, functions, and methods usable in combination therewith should or must be excluded.

What is claimed is:

1. A method, comprising:
determining a target dose amount of medicament to deliver for a control cycle;
obtaining data related to an extended bolus;
determining an additional medicament amount to deliver during the control cycle corresponding to the extended bolus;
determining a total medicament delivery amount for the control cycle corresponding to the target dose amount and the additional medicament amount; and
sending a command to a delivery mechanism to cause delivery of the total medicament delivery amount for the control cycle.

2. The method of claim 1, wherein the total medicament delivery amount is determined by adding the target dose amount for the control cycle to the additional medicament amount for the control cycle, where the additional medicament amount is an amount of the extended bolus for the control cycle.

3. The method of claim 1, wherein the additional medicament amount corresponding to the extended bolus is only delivered during the control cycle if the target dose amount of medicament is above a threshold value.

4. The method of claim 3, wherein the threshold value is based on a current basal medicament delivery rate for a user of an automated medicament delivery system.

5. The method of claim 4, wherein the threshold value is a basal amount of medicament delivered per hour divided by a number of control cycles per hour.

6. The method of claim 1, wherein:
the target dose amount of the medicament to deliver for the control cycle is determined by an algorithm executed by a controller for an automated medicament delivery system;
the extended bolus is incorporated into an input for the algorithm with a basal amount; and
determining the target dose amount of the medicament to deliver for the control cycle and determining the additional medicament amount to deliver during the control cycle corresponding to the extended bolus are performed simultaneously.

7. The method of claim 6, wherein constraints for medicament delivery in the algorithm are scaled based on the extended bolus incorporated into the input.

8. The method of claim 1, further comprising applying an offset to safety constraints applied to medicament delivery to ensure safe and efficient delivery of the extended bolus with the target dose amount.

9. The method of claim 8, further comprising reversing the offset after each respective constraint calculation.

10. The method of claim 1, wherein the data includes an amount of medicament and a duration of the extended bolus or information related to determining the extended bolus.

11. An automated medicament delivery device, comprising:
one or more processors; and
memory including instructions, when executed, cause the one or more processors to:
determine a target dose amount of medicament to deliver for a control cycle;
obtain data related to an extended bolus;
determine an additional medicament amount to deliver during the control cycle corresponding to the extended bolus;
determine a total medicament delivery amount for the control cycle corresponding to the target dose amount and the additional medicament amount; and
deliver the total medicament amount during the control cycle utilizing an automated medicament delivery system.

12. The automated medicament delivery device of claim 11, wherein the total medicament delivery amount is determined by adding the target dose amount for the control cycle to the additional medicament amount for the control cycle, where the additional medicament amount is an amount of the extended bolus for the control cycle.

13. The automated medicament delivery device of claim 11, wherein the additional medicament amount corresponding to the extended bolus is only delivered during the control cycle if the target dose amount of medicament is above a threshold value.

14. The automated medicament delivery device of claim 13, wherein the threshold value is based on a current basal medicament delivery rate for a user of the automated medicament delivery system.

15. The automated medicament delivery device of claim 14, wherein the threshold value is a basal amount of medicament delivered per hour divided by a number of control cycles per hour.

16. The automated medicament delivery device of claim 11, wherein:
the target dose amount of the medicament to deliver for the control cycle is determined by an algorithm executed by a controller for the automated medicament delivery system;
the extended bolus is incorporated into an input for the algorithm with a basal amount; and
determining the target dose amount of the medicament to deliver for the control cycle and determining the additional medicament amount to deliver during the control cycle corresponding to the extended bolus are performed simultaneously.

17. The automated medicament delivery device of claim 16, wherein constraints for medicament delivery in the algorithm are scaled based on the extended bolus incorporated into the input.

18. The automated medicament delivery device of claim 11, wherein the memory including the instructions, when executed, cause the one or more processors to apply an offset to safety constraints applied to medicament delivery to ensure safe and efficient delivery of the extended bolus with the target dose amount.

19. The automated medicament delivery device of claim 18, wherein the memory including the instructions, when executed, cause the one or more processors to reverse the offset after each respective constraint calculation.

20. The automated medicament delivery device of claim 11, wherein the data includes an amount of medicament and a duration of the extended bolus or information related to determining the extended bolus.
